(19) United States
(12) Patent Application Publication
(10) Pub. No.: US 2026/0152461 A1
(43) Pub. Date: Jun. 4, 2026

(54) PREPARATION OF ALPHA-NECRODYL ISOBUTYRATE FROM DIENES

(71) Applicant: Suterra, LLC, Los Angeles, CA (US)

(72) Inventors: Donald Raymond Wenz, Bend, OR (US); Eric Allen Bercot, Bend, OR (US)

(21) Appl. No.: 18/713,029

(22) PCT Filed: Nov. 23, 2022
(86) PCT No.: PCT/US2022/050900

§ 371 Date: May 23, 2024

Related U.S. Application Data
(60) Provisional application No. 63/282,459, filed on Nov. 23, 2021.

(51) Int. Cl. C07C 67/293; C07C 29/10; C07C 29/147; C07C 33/025; C07C 33/035; C07C 41/09; C07C 43/178; C07C 45/29; C07C 47/277; C07C 67/347; C07C 67/36; C07C 67/475; C07C 69/24; C07C 69/604

(52) U.S. Cl. CPC C07C 67/293; C07C 29/10; C07C 29/147; C07C 33/025; C07C 33/035; C07C 41/09; C07C 43/178; C07C 45/29; C07C 47/277; C07C 67/347; C07C 67/36; C07C 67/475; C07C 69/24; C07C 69/604

(57) ABSTRACT

Described herein are methods of preparing α-necrodyl isobutryate that comprise cyclizing a compound of formula (IX): to produce a mixture of the compounds of formulas (Xa) and (Xb); then reacting the mixture of compounds of formulas (Xa) and (Xb) with an isobutyryl donor, to produce α-necrodyl isobutyrate.

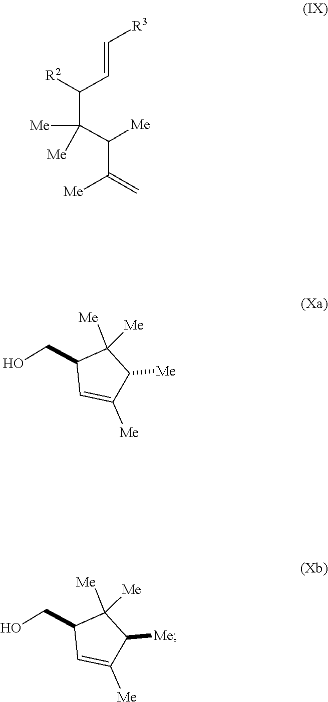

### CROSS-REFERENCE TO RELATED APPLICATIONS

This application claims priority to U.S. Provisional Patent Application No. 63/282,459 filed on Nov. 23, 2021, the entire disclosure of which is hereby incorporated by reference in its entirety.

### TECHNICAL FIELD

This disclosure relates generally to methods of preparing α-necrodyl isobutryate from diene intermediate compounds.

### INTRODUCTION

Grape mealybug (Pseudococcus maritimus) is a pest of grapes and pome fruits in the United States, Europe and South America. Grape mealybugs feed on plant fluids causing fruit damage, impacting long term plant viability and acting as a disease vector. For these reasons, the control of grape mealybug is of great interest to commercial growers. Current grape mealybug control methods typically rely on wide spectrum insecticide sprays that suffer from residue and long-term resistance issues making the development of pheromone-based control products of significant commercial interest.

The sex pheromone of the grape mealybug (Pseudococcus maritimus) was identified as trans-α-necrodyl isobutryate by Millar and co-workers in 2007 (Figadere, B. A. et. al. Tetrahedron Lett. 2007, 48, 8434-8437). A subsequent follow up study by the same group in 2010 showed that a racemic mixture of trans-α-necrodyl isobutryate was more effective than either enantiomer alone in attracting grape mealybug in a field setting.

### SUMMARY

In some aspects, the present disclosure provides methods of preparing α-necrodyl isobutyrate, the methods comprising:
(i) cyclizing a compound of formula (IX):

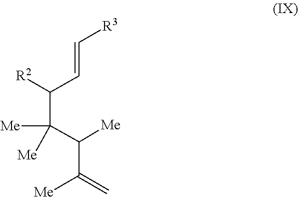

wherein:

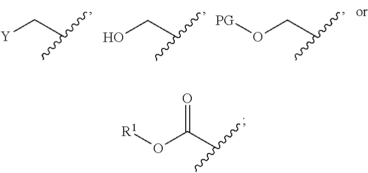

R² is
Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl; and

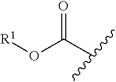

when R² is a reducing agent is added pre-or post-cyclization;
R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, —C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents;
R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂;
to produce a mixture of the compounds of formulas (Xa) and (Xb):

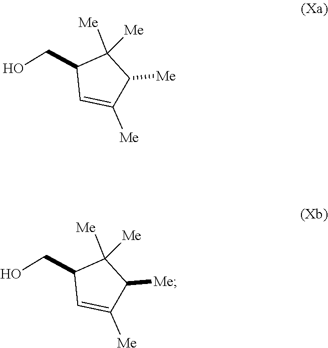 [SMILES: CC1=C[C@@H](CO)C(C)(C)[C@@H]1C ; CC1=C[C@@H](CO)C(C)(C)[C@H]1C]

then
(ii) reacting the mixture of compounds of formulas (Xa) and (Xb) with an isobutyryl donor, to produce α-necrodyl isobutyrate; or, alternatively,
cyclizing a compound of formula (IX) wherein R² is

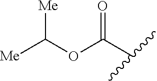 [SMILES: *C(=O)OC(C)C]

to produce
   α-necrodyl isobutyrate.

In some aspects, the methods may further comprise olefinating a compound of formula (VII):

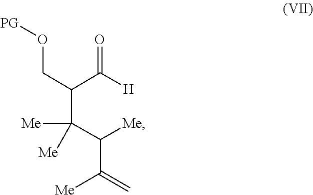

and removing the hydroxyl protecting group (PG), to produce compounds of formula (IX) where R² is

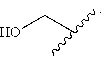 [SMILES: *CO]

The methods may further comprise oxidizing compounds of formula (VI):
to produce compounds of formula (VII).

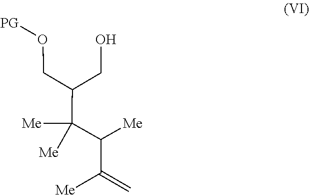

The methods may further comprise adding a hydroxyl protecting group to a compound of formula (V):
to produce a compound of formula (VI).

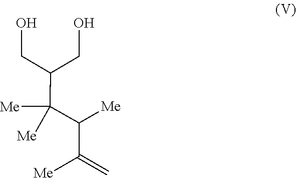

The methods may further comprise reducing a compound of formula (III):

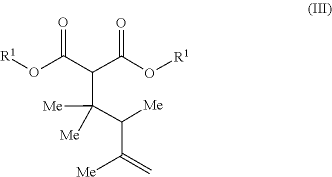

with a reducing agent to produce a compound of formula (V).

Alternatively, in other aspects the methods of preparing α-necrodyl isobutyrate may comprise olefinating a compound of formula (VIII):

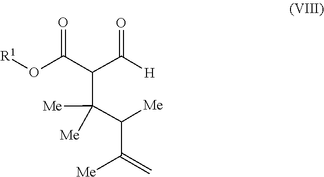

to produce a compound of formula (IX) where R² is

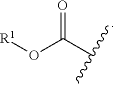

The methods may further comprise formylating a compound of formula (IV):

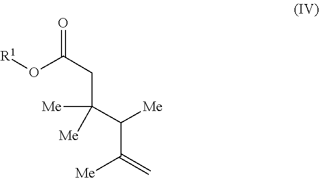

to produce a compound of formula (VIII).

The methods may further comprise selectively decarboxylating one

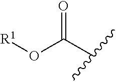

moiety in a compound of formula (III):

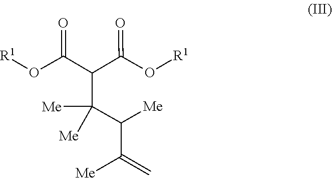

to produce a compound of formula (IV).

The methods may additionally comprise reacting a compound of formula (I), wherein X is chlorine, bromine, or iodine, with a compound of formula (II):

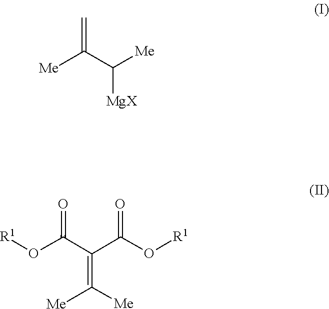

to a produce a compound of formula (III).

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### I. Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,”, “may,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” is used to indicate that exact values are not necessarily attainable. Therefore, the term “about” is used to indicate this uncertainty limit. The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5-1.4. The modifier “about” should also be considered as disclosing the range defined by the absolute values of the two endpoints. For example, the expression “from about 2 to about 4” also discloses the range “from 2 to 4.”

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkoxy,” as used herein, refers to a group-O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means-NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9-to 12-membered fused bicyclic system).

The term “cyanoalkyl,” as used herein, means at least one-CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

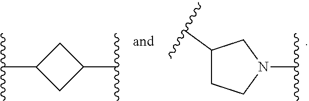 [SMILES: *C1CC(*)C1 ; *C1CCN(*)C1]

Cycloalkylene and heterocyclylene include a geminal divalent groups such as 1,1-C₃₋₆cycloalkylene (i.e.,

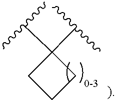

A further example is 1,1-cyclopropylene (i.e.,

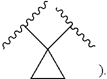 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8-to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven-and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### II. Diene Intermediate Compounds

In some aspects, the present disclosure provides diene intermediate compounds of formulas (III) (IV), (VIII)-(IX), and (XI), wherein R¹, R², R³, R⁴, and R⁵ are as defined herein.

Diene intermediate compounds useful in the present disclosure are set forth in the following numbered embodiments. The first embodiment is denoted E1, the second embodiment is denoted E2 and so forth.

E1. A compound of formula (IX):

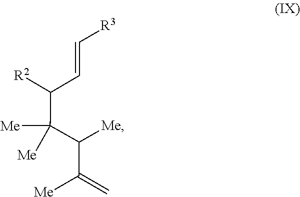

wherein:
R² is

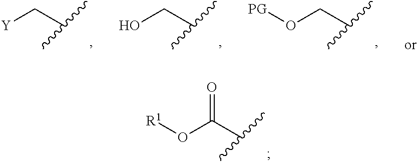

Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl;

R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, —C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents; and

R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂.

E2. The compound of embodiment 1, wherein R³ is C₁alkyl substituted with 1 substituent selected from the group consisting of

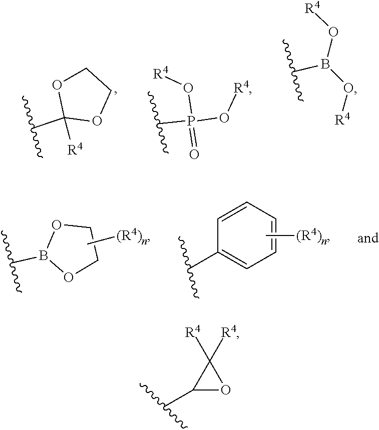

where n is 0, 1, 2, 3, or 4.

E3. The compound of embodiment 1, wherein R³ is hydrogen.

E4. A compound of formula (XI):

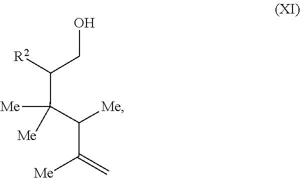

wherein:
R² is

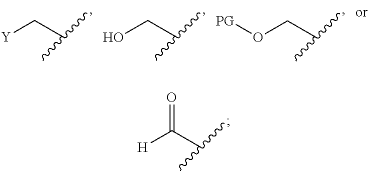

and
Y is a halogen;
PG is a hydroxyl protecting group.

E5. The compound of embodiment 4, wherein the hydroxyl protecting group is a tert-butyl(dimethyl) silyl (TBS) group.

E6. A compound of formula (VIII):

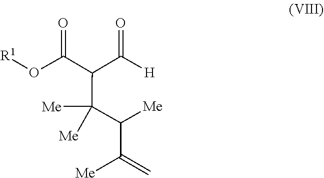

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E7. A compound of formula (IV):

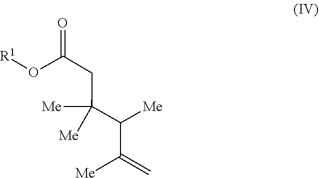

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E8. A compound of formula (III):

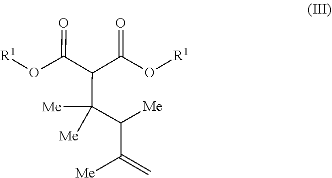

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E9. The compound of any one of embodiments 1-2 or 6-8 wherein R¹ is methyl or ethyl.

### III. Methods for Preparing α-Necrodyl Isobutyrate

α-Necrodyl isobutyrate may be prepared by exemplary synthetic processes depicted in the following schemes.

Abbreviations which have been used in the descriptions of the Schemes that follow are:
Me is methyl;
Et is ethyl;
Cp is η⁵-cyclopentadienyl;
Ph is phenyl;
Ac is acetyl;
Bn is benzyl;
KO^{t}Bu is potassium tert-butoxide;
Ms is methane sulfonyl;
Ts is toluene sulfonyl;
Tf is trifluoromethane sulfonate;
TMS is trimethyl silane;
TBS is tert-butyl(dimethyl) silyl;
TBDPS is tert-butyl(diphenyl) silyl;
TIPS is triisopropylsilyl;
NEt₃ is triethylamine;
n-BuLi is n-butyl lithium;
LDA is lithium diisopropyl amide;
LiHMDS is lithium bis(trimethylsilyl)amide;
KHMDS is potassium bis(trimethylsilyl)amide;
NaHMDS is sodium bis(trimethylsilyl)amide;
Δ is heat;
MHₓ is a metal hydride;
PG is protecting group;
DIBAL-H is diisobutylaluminum hydride;
DMSO is dimethyl sulfoxide;
PPh₃ is triphenyl phosphine;
PhMe is toluene;
THF is tetrahydrofuran; and
Hoveyda-Grubbs Catalyst is (1,3-Bis-(2,4,6-trimethylphenyl)-2-imidazolidinylidene)dichloro(o-isopropoxyphenylmethylene) ruthenium

In some aspects of the present disclosure, α-necrodyl isobutyrate may be synthesized according to the sequence depicted in General Scheme A.

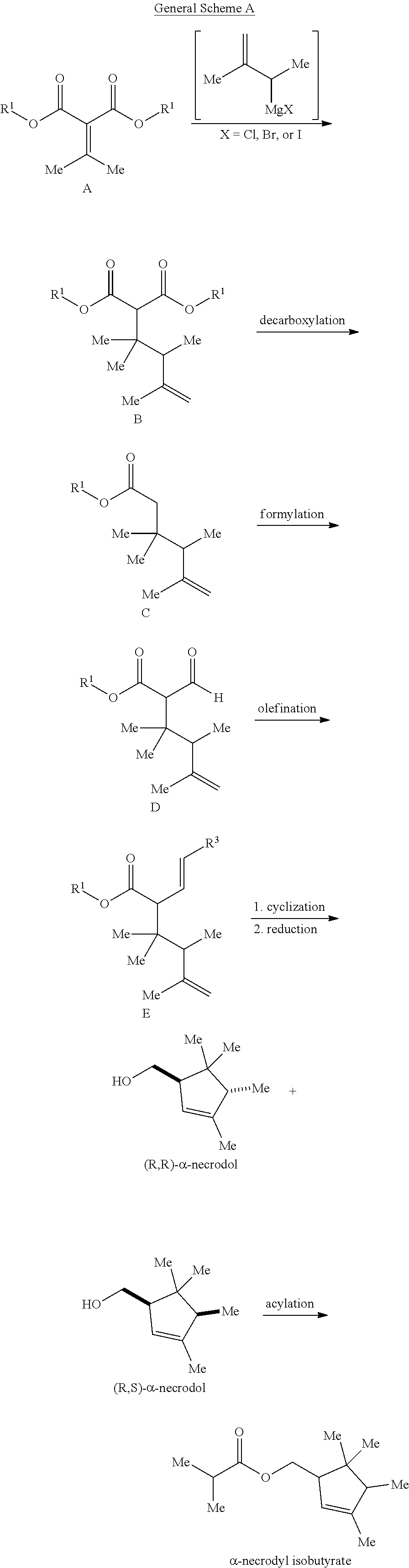

In other aspects of the present disclosure, α-necrodyl isobutyrate may be synthesized according to the sequence depicted in General Scheme B.

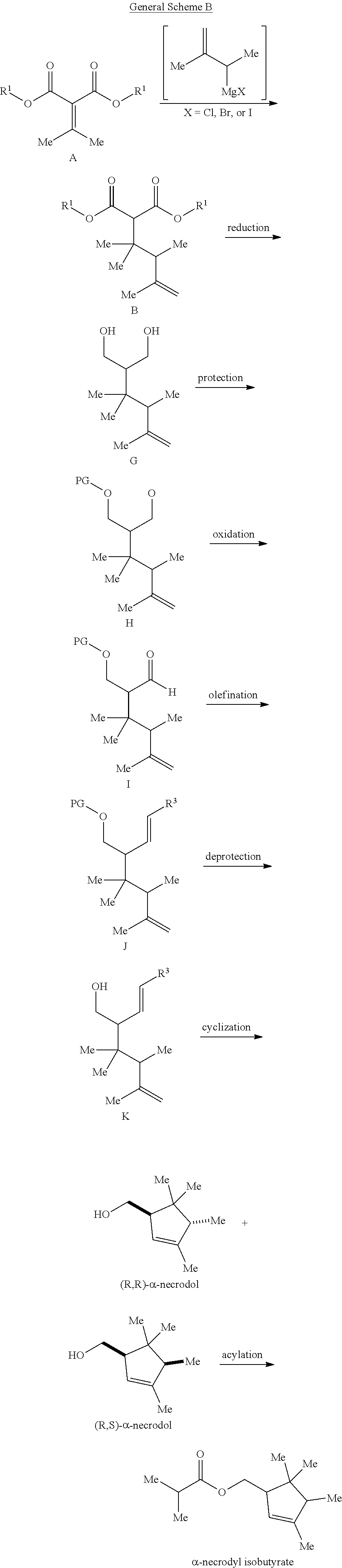

Each transformation shown in the sequences above will be described in more detail below.

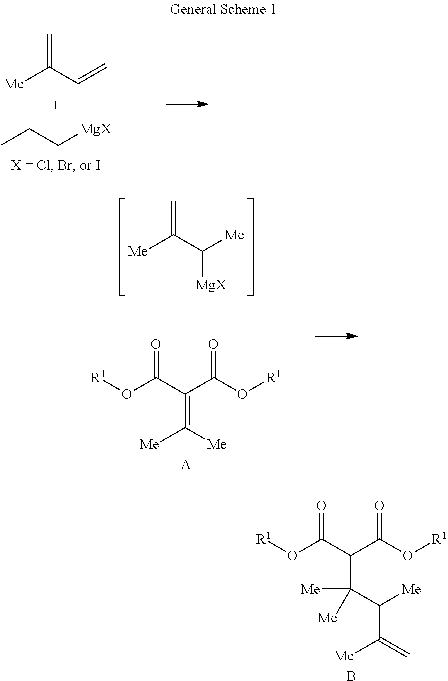

General Scheme 1 depicts the first step in General Schemes A and B. As shown in General Scheme 1, a γ,γ-malonate of formula A may be reacted with an allylic Grignard reagent to form an intermediate diester compound of formula B.

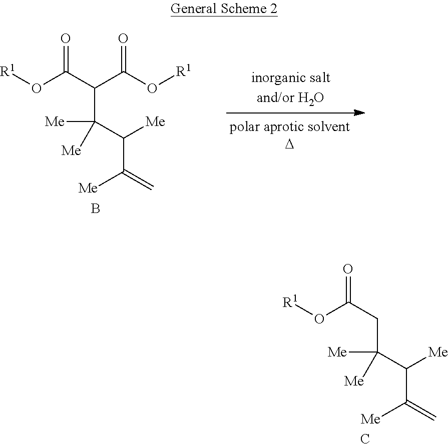

General Scheme 2 depicts the second step in General Scheme A. As shown in General Scheme 2, an intermediate compound of formula B may be reacted with an inorganic salt under standard decarboxylation reaction conditions to form a monoester intermediate compound of formula C. In various instances, the inorganic salt may be LiCl. In various instances, the polar aprotic solvent may be DMSO. In various instances, the reaction may be heated to at least 150° C.

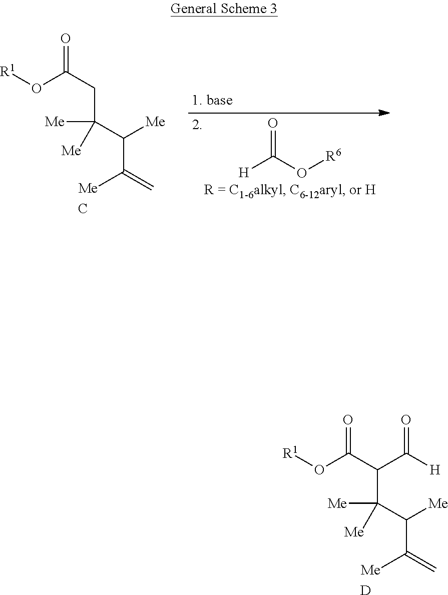

General Scheme 3 depicts the third step in General Scheme A. As shown in General Scheme 3, intermediate compounds of formula C may be formylated under standard formylation conditions. In various instances, a compound of formula C is reacted with a base followed by a formate or formic acid to form a compound of formula D. Suitable bases include, but are not limited to, organometallic bases (e.g., lithium bis(trimethylsilyl)amide (LiHMDS), potassium bis(trimethylsilyl)amide (KHMDS), sodium bis(trimethylsilyl)amide (NaMDS), or lithium dialkyl amide base (e.g., lithium diisopropylamide (LDA)), or potassium tert-butoxide (KO^{t}Bu). In various instances, a compound of formula C may be reacted with a base followed by ethyl formate (HCO₂Et) to form a compound of formula D. In various instances, the base may be an organolithium base. The organolithium base may be a lithium amide base (e.g., LiHMDS) or a lithium dialkyl amide base (e.g., LDA). In various instances, the base may be LDA.

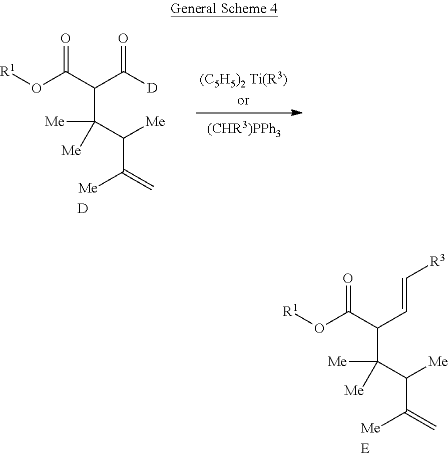

General Scheme 4 depicts the fourth step in General Scheme A. As shown in General Scheme 4, intermediate compounds of formula D may be olefinated by reacting a compound of formula D with a titanium methylidene or with a phosphonium ylide (e.g., a phosphonium ylide of formula (CHR³) PPh₃) to form an intermediate compound of formula E. In various instances, the compound of formula D is reacted with a titanium methylidene of formula (C₅H₅)₂Ti(CHR³). In various instances, the compound of formula D is reacted with a phosphonium ylide of formula (CHR³) PPh₃. In various instances, the titanium methylidene may be (C₅H₅)₂Ti(CH₂). In various instances, the phosphonium ylide may be (CH₂) PPh₃. Titanium methylidene and phosphonium ylide reagents may be prepared using known procedures. For example, titanium methylidenes of formula (C₅H₅)₂Ti((CHR³) may be prepared by reacting a reagent of formula (C₅H₅): Ti(CHR³)ClAl(CH₃)₂ (e.g., Tebbe's reagent) or a reagent of formula Cp₂Ti(CH₂R³)₂ (e.g., Petasis reagent) with a mild Lewis base (e.g., pyridine). Phosphonium ylides may be prepared from phosphonium salts by deprotonation with an appropriate base (e.g., KO^{t}Bu or n-BuLi). Phosphonium salts may be prepared by reacting alkyl halides (e.g. alkyl bromides of formula CH₂R³Br) and triphenylphosphine (PPh₃).

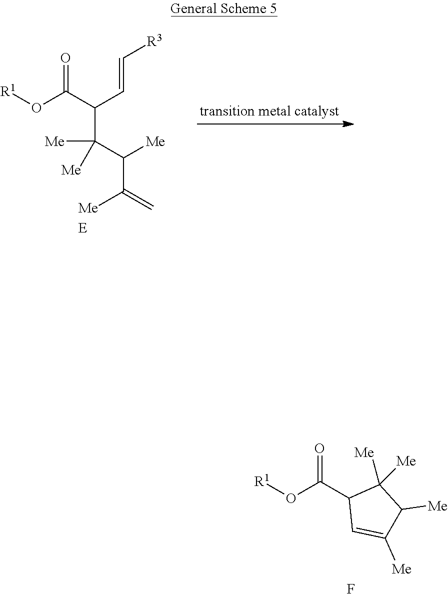

General Scheme 5 depicts the fifth step in General Scheme A. As shown in General Scheme 5, intermediate compounds of formula E may be cyclized via olefin cross metathesis (also referred to as ring-closing metathesis (RCM)). Intermediate compounds of formula E may be cyclized by reacting a compound of formula E with a transition metal catalyst (e.g., a transition metal carbene complex catalyst) to form an intermediate compound of formula F. Exemplary solvents for the reaction include aprotic organic solvents (e.g., toluene). In various instances, the transition metal catalyst is a ruthenium (II) carbene complex catalyst. In various instances the ruthenium (II) carbene complex catalyst is (1,3-bis(2,4,6-trimethylphenyl)-2-imidazolidinylidene)dichloro(o-isopropoxyphenylmethylene) ruthenium.

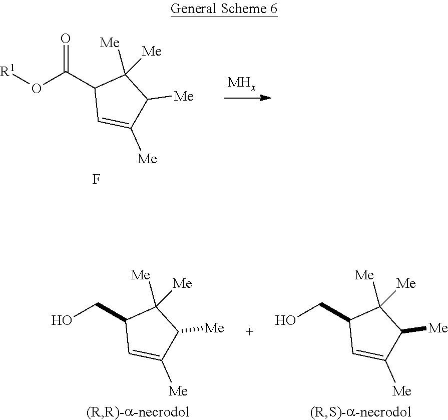

General Scheme 6 depicts the penultimate step in General Scheme A. As shown in General Scheme 6, intermediate compounds of formula F may be reduced to produce a diastereomeric mixture of (R,R)-α-necrodol and (R,S)-α-necrodol, by reacting a compound of formula F with a metal hydride (MHₓ) reducing agent. Example metal hydride reducing agents are depicted in Table 1.

TABLE 1
Example metal hydride (MHₓ) reducing agents
MHₓ

| | |
|---|---|
| 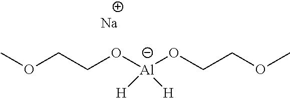 | sodium bis(2- methoxyethoxy)aluminum hydride |
| 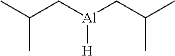 | diisobutyl aluminum hydride |
| 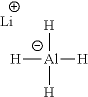 | lithium aluminum hydride |
| 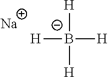 | sodium borohydride |

In various instances, the reducing agent may be lithium aluminum hydride (LiAlH₄), sodium borohydride (NaBH₄), sodium bis(2-methoxyethoxy)aluminum hydride, or diisobutylaluminum hydride (DIBAL-H) (Table 1). In various instances, the reducing agent is sodium bis(2-methoxyethoxy)aluminum hydride.

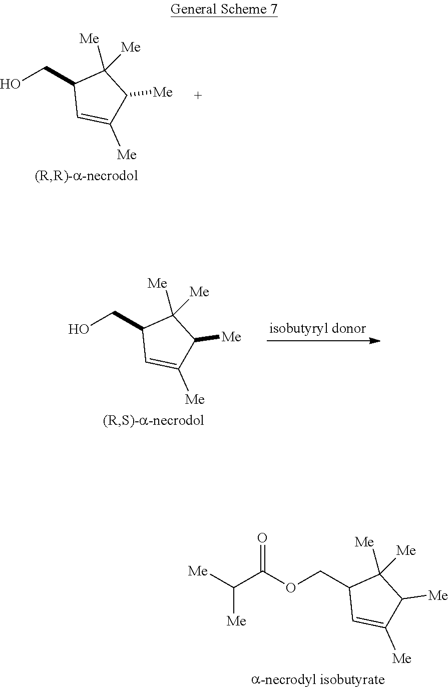

General Scheme 7 depicts the final step in General Schemes A and B. A diastereomeric mixture of (R,R)-α-necrodol and (R,S)-α-necrodol may be reacted with an isobutyryl donor to form α-necrodyl isobutyrate. Suitable isobutyryl donors include, but are not limited to, isobutyryl chloride, isobutyric acid, isobutyric anhydride, and alkyl isobutyrates (e.g., methyl, ethyl, or propyl isobutyrate). The mixture of (R,R)-α-necrodol and (R,S)-α-necrodol may be reacted with the isobutyryl donor under suitable acylation conditions. For example (R,R)-α-necrodol and (R,S)-α-necrodol may be reacted with the isobutyryl donor in the presence of 4-(dimethylamino)pyridine (DMAP) and triethylamine (NEt₃) in an organic solvent. The organic solvent may be methyl tert-butyl ether. In various instances, the isobutyryl donor may be

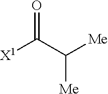

wherein X¹ is Cl, Br, I, —OH, —OC₁₋₄alkyl, —OPiv,

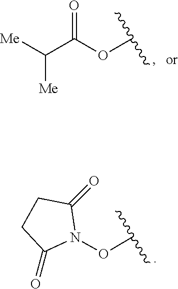 [SMILES: *OC(=O)C(C)C ; *ON1C(=O)CCC1=O]

In various instances the isobutyryl donor may be isobutyryl chloride.

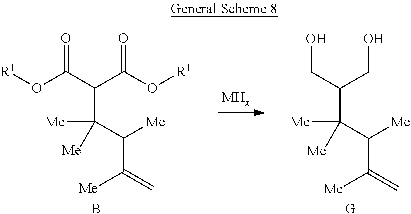

General Scheme 8 depicts the second step in General Scheme B. General Scheme 8 shows the reduction of diester intermediate compounds of formula B to diol intermediate compounds of formula G. A compound of formula B may be reacted with a metal hydride (MHₓ) to produce a compound of formula G. Example metal hydride reducing agents are depicted in Table 1. In various instances, the reducing agent may be lithium aluminum hydride (LiAlH₄), sodium borohydride (NaBH₄), sodium bis(2-methoxyethoxy)aluminum hydride, or diisobutylaluminum hydride (DIBAL-H) (Table 1). In various instances, the reducing agent is sodium bis(2-methoxyethoxy)aluminum hydride.

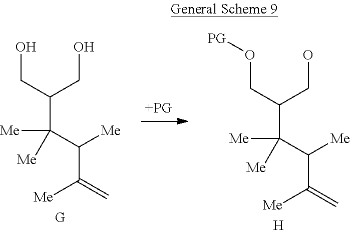

General Scheme 9 depicts the third step in General Scheme B. General Scheme 9 shows the monoprotection of diol intermediates of formula G to form mono-protected compounds of formula H. Protection of a hydroxyl group for compounds of formula G may be by any appropriate method described in Greene and Wuts, or other methods well known to those skilled in the art for the desired protecting group (PG). For example, if the protecting group (PG) is a silyl ether, the hydroxyl group protection may comprise reacting a compound of formula G with a silylating agent, such as chlorotrimethylsilane (TMSCI), tert-butyldiphenylsilyl chloride tert-butyl(diphenyl)silyl (TBDPSCI), triisopropylsilyl chloride (TIPSCI), or tert-butyl(dimethyl)silyl chloride (TBSCLl), in the presence of a base and an organic solvent to form a silylated compound of formula H. Exemplary organic solvents include aprotic solvents, such as tetrahydrofuran (THF), dimethyl sulfoxide (DMSO), and toluene (PhMe). Suitable bases include imidazole, triethylamine, pyridine, sodium hydride (NaH), and potassium tert-butoxide (KO^{t}Bu). In various instances, the silylating agent may be TBSCLl, the base may comprise potassium tert-butoxide (KO^{t}Bu), and the solvent may comprise tetrahydrofuran (THF). In various instances, the solvent may further comprise toluene (PhMe). In various instances, the protecting group (PG) in the resulting compounds of formula H may be trimethylsilyl (TMS), tert-butyl(diphenyl) silyl (TBDPS), triisopropylsilyl (TIPS), or tert-butyl(dimethyl) silyl (TBS). In various instances, the protecting group (PG) is tert-butyl(dimethyl) silyl (TBS).

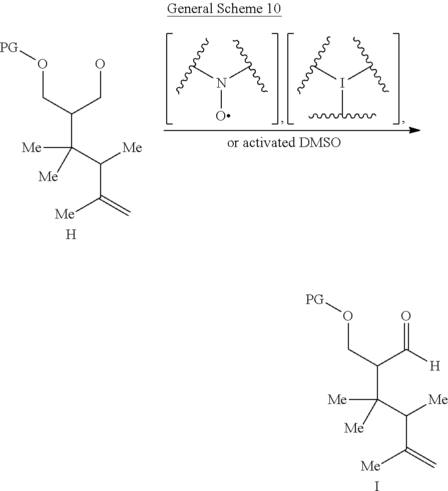

As shown in General Scheme 10, intermediate alcohol compounds of formula H may be oxidized by reacting a compound of formula H with an oxidizing agent to form a compound of formula I. Suitable oxidizing agents include, but are not limited to, nitroxyl radicals, hypervalent iodine compounds, and activated DMSO (may be formed by reacting DMSO with an activating agent, including, but not limited to, oxalyl chloride, SO₃, pyridine, or acetic anhydride). Table 2 shows examples of nitroxyl radicals that may be used to oxidize compounds of formula H.

TABLE 2
Example Nitroxyl Radicals

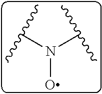

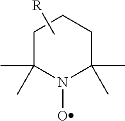

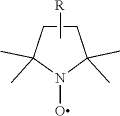

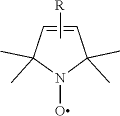

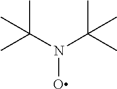

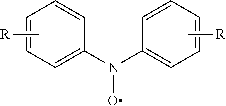

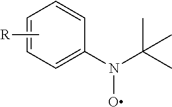

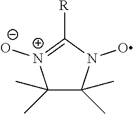

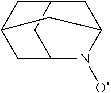

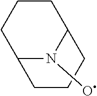

R = H, OH, NH₂, C(O)NH₂, Me, OMe, Ph, or

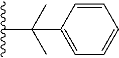 [SMILES: *C(C)(C)c1ccccc1]

Table 3 shows examples of hypervalent iodine compounds that may be used to oxidize compounds of formula H.

TABLE 3
Example Hypervalent Iodine Compounds

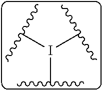 [SMILES: *I(*)*]

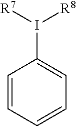

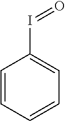

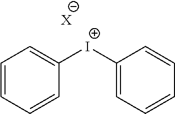

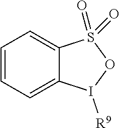

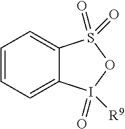

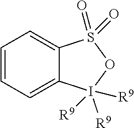

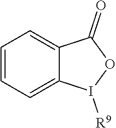

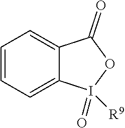

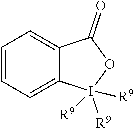

R⁷, R⁸ = —OH, —OAc, —OBn, —OMs, —OTf, Cl, F, Ph, —OC₁₋₆alkyl, —OCF₃, —OC(O)C₁₋₆alkyl, or —OC(O)CF₃
R⁹ = OAc, OH, F, Cl, OC₁₋₆alkyl, or —OCF₃
X = F, Cl, Br, or I

In various instances, the nitroxyl radical is formed by reacting a nitroxyl radical precursor with a stoichiometric oxidant, where the nitroxyl radical precursor is (2,2,6,6-tetramethylpiperidin-1-yl)oxyl (TEMPO), 4-hydroxy-(2,2,6,6-tetramethylpiperidin-1-yl)oxyl4-acetamido (4-OH-TEMPO), 4-acetamido-(2,2,6,6-tetramethylpiperidin-1-yl)oxyl (4-acetamido-TEMPO), 2-azaadamantane N-oxyl (AZADO), or 9-azabicyclo[3.3.1]nonane N-oxyl (ABNO). In various instances, the nitroxyl radical precursor is TEMPO. In various instances, the stoichiometric oxidant is sodium hypochlorite, oxygen, or (diacetoxyiodo)benzene.

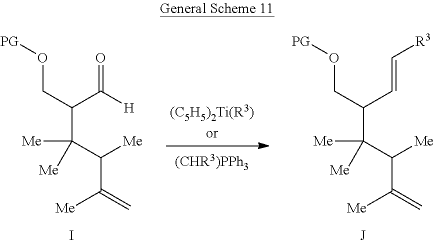

General Scheme 11 depicts the fifth step in General Scheme B. As shown in General Scheme 11, intermediate compounds of formula I may be olefinated by reacting a compound of formula I with a titanium methylidene or with a phosphonium ylide (e.g., a phosphonium ylide of formula (CHR³) PPh₃) to form an intermediate compound of formula J. In various instances, the compound of formula I is reacted with a titanium methylidene of formula (C₅H₅)₂Ti(CHR³). In various instances, the compound of formula I is reacted with a phosphonium ylide of formula (CHR³) PPh₃. In various instances, the titanium methylidene may be (C₅H₅)₂Ti(CH₂). In various instances, the phosphonium ylide may be (CH₂) PPh₃. Titanium methylidene and phosphonium ylide reagents may be prepared using known procedures. For example, titanium methylidenes of formula (C₅H₅)₂Ti((CHR³) may be prepared by reacting a Tebbe reagent of formula (C₅H₅): Ti(CHR³)ClAl(CH₃)₂ or a Petasis reagent of formula Cp₂Ti(CH₂R³)₂ with a mild Lewis base (e.g., pyridine). Phosphonium ylides may be prepared from phosphonium salts by deprotonation with an appropriate base (e.g., KO^{t}Bu or n-BuLi). Phosphonium salts may be prepared by reacting alkyl halides (e.g. alkyl bromides of formula CH₂R³Br) and triphenylphosphine (PPh₃).

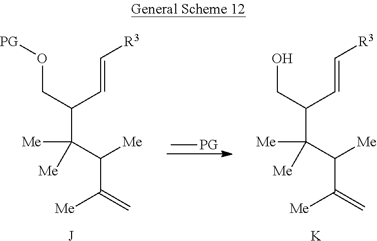

As shown in General Scheme 12, protected alcohol intermediate compounds of formula J may be deprotected to form alcohol intermediate compounds of formula K. Deprotection of a protected hydroxyl group in a compound of formula J may be by any appropriate method described in Greene and Wuts, or other methods well known to those skilled in the art for the particular protecting group. For example, if the protecting group (PG) is a silyl ether, then reacting a protected hydroxyl group of formula J with an acid or fluoride would be a suitable method for converting the protected alcohol of formula J to an alcohol of formula K. Thus, in some implementations, the deprotection is by reacting the protected alcohol with an acid (e.g., p-toluene sulfonic acid) or a fluoride (e.g., tetra-n-butylammonium fluoride (TBAF)). The particular deprotection method is selected according to the requirements of the particular protecting group and other functionality present in the molecule.

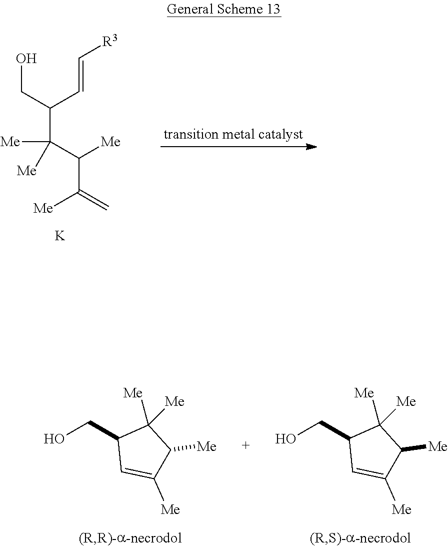

General Scheme 13 depicts the penultimate step in General Scheme B. As shown in General Scheme 13, intermediate compounds of formula K may be cyclized via olefin cross metathesis (also referred to as ring-closing metathesis (RCM)). Intermediate compounds of formula K may be cyclized by reacting a compound of formula K with a transition metal catalyst (e.g., a transition metal carbene complex catalyst) to produce a diastereomeric mixture of (R,R)-α-necrodol and (R,S)-α-necrodol. Exemplary solvents for the reaction include aprotic organic solvents (e.g., toluene). In various instances, the transition metal catalyst is a ruthenium (II) carbene complex catalyst. In various instances the ruthenium (II) carbene complex catalyst is (1,3-bis(2,4,6-trimethylphenyl)-2-imidazolidinylidene)dichloro(o-isopropoxyphenylmethylene) ruthenium.

Olefination conditions suitable for use in the processes of General Scheme 4 and General Scheme 11 are well known in the art. Suitable conditions include those generally outlined in General Scheme 4 and General Scheme 11, and as described in the Examples herein.

Reduction conditions suitable for use in the processes of General Scheme 6 and General Scheme 8 are well known in the art. Suitable conditions include those generally outlined in General Scheme 6 and General Scheme 8, and as described in the examples herein.

Cyclization conditions suitable for use in the processes of General Scheme 7 and General Scheme 13 are well known in the art. Suitable conditions include those generally outlined in General Scheme 7 and General Scheme 13, and as described in the Examples herein.

α-Necrodyl isobutyrate and the intermediate compounds may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a disclosed compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### IV. EXAMPLES

The foregoing may be better understood by reference to the following examples, which are presented for purposes of illustration and are not intended to limit the scope of the technology.

### Abbreviations

aq. is aqueous;
Me is methyl;
Et is ethyl;
g is gram(s);
kg is kilogram(s);
L is liter(s);
mol or mol. is mole(s);
M is mole(s) per liter;
N is number of mole equivalent(s) per liter;
wt % or % (w/w) is weight percent;
eq, eq., or equiv is equivalent(s);
sat. is saturated;
h or hr is hour(s);
min or min. is minute(s);
s or sec is second(s);
DMSO is dimethyl sulfoxide;
THF is tetrahydrofuran;
TBS is tert-butyl(dimethyl) silyl;
GCMS is gas chromatography mass spectrometry;
NMR is nuclear magnetic resonance;
bp is boiling point; and
Cl is chemical ionization.

### Example 1: Preparation of diethyl 2-(2,3,4-trimethylpent-4-en-2-yl) malonate

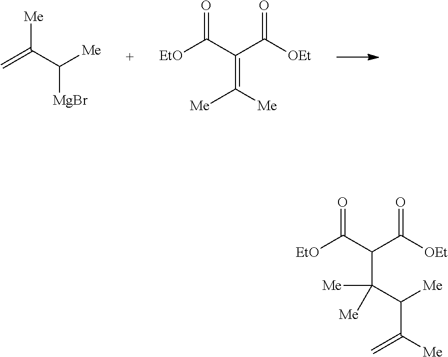

A clean and dry reactor under N₂ atmosphere was charged with magnesium turnings (875 g, 36.03 mol), 2-methyltetrahydrofuran (10.0 L) and toluene (10.0 L). Next, 1-bromopropane (4.387 kg, 135.67 mol) was added to the reactor. The reaction was stirred until conversion to propylmagnesium bromide was complete. Titanocene dichloride (88.81 g, 0.36 mol) was then added to the reactor. The mixture was warmed to 40° C., then isoprene (3.038 kg, 44.59 mol) was added, and the reaction was aged until conversion to the allyl Grignard reagent was complete. The mixture was cooled to −10° C. and diethyl isopropylidenemalonate (5.00 kg, 24.97 mol) was added. The reaction was aged then quenched with 20% (w/w) citric acid (17.5 L). The organic phase was subsequently washed with sat. aq. NaHCO₃ and water. The organic phase was collected and concentrated in vacuo producing 7.151 kg (85% yield) of desired product as an oil. 1H NMR (600 MHz, CDCl₃) δ ppm 0.97-1.05 (m, 6H)1.12 (s, 3H)1.20-1.26 (m, 6H)1.70 (s, 3H)2.61 (q, J=7.15 Hz, 1H)3.47 (s, 1H)4.10-4.18 (m, 4H)4.67-4.71 (m, 1H)4.81 (s, 1H). GCMS (Cl) m z 271.0 (270.18 calcd. for C₁₇H₃₄O₂⁺ [M]⁺).

### Example 2: Preparation of 2-(2,3,4-trimethylpent-4-en-2-yl) propane-1,3-diol

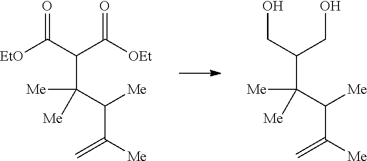 [SMILES: C=C(C)C(C)C(C)(C)C(C(=O)OCC)C(=O)OCC ; C=C(C)C(C)C(C)(C)C(CO)CO]

A clean and dry reactor under N₂ atmosphere was charged with toluene (27.7 L) and a 70% (w/w) solution of sodium bis(2-methoxyethoxy)aluminum hydride in toluene (13.969 kg, 48.37 mol). Diethyl 2-(2,3,4-trimethylpent-4-en-2-yl) malonate (5.030 kg, 18.60 mol) was added and aged at 20° C. until reaction deemed complete. The reaction was cooled to 10° C., then 20% (w/w) aq. NaOH was added. Water was added following caustic addition, then the batch was stirred until all solids had dissolved. The aqueous phase was removed, then the organic phase was washed three times with water. The solvent was removed in vacuo and the title compound was collected and purified by distillation under vacuum (bp 93° C./0.4 torr) to afford 2.997 kg (86% yield) of the title compound as a waxy solid. 1H NMR (600 MHZ, CDCl₃) δ ppm 0.85 (s, 3H)0.87 (s, 3H)1.03 (d, J=6.97 Hz, 3H)1.77 (s, 3H)1.90 (ddd, J=8.99, 5.69, 3.30 Hz, 1H)2.33 (q, J=7.21 Hz, 1H) 2.79 (br s, 1H)2.83 (br s, 1H)3.80 (dt, J=19.99, 9.81 Hz, 2H)4.00-4.06 (m, 2H)4.74 (s, 1H) 4.88 (s, 1H). GCMS (Cl) m z 186.9 (186.16 calcd. for C₁₁H₂₂O₂⁺ [M]⁺).

### Example 3: Preparation of 2-(((tert-butyldimethylsilyl)oxy)methyl)-3,3,4,5-tetramethylhex-5-en-1-ol

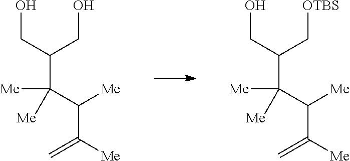 [SMILES: C=C(C)C(C)C(C)(C)C(CO)CO ; C=C(C)C(C)C(C)(C)C(CO)CO[Si](C)(C)C(C)(C)C]

A clean and dry reactor under N₂ atmosphere was charged with a 20% (w/w) solution of potassium tert-butoxide in THF (8.132 kg, 14.50 mol), then 2-(2,3,4-trimethylpent-4-en-2-yl) propane-1,3-diol (2.702 kg, 14.50 mol) and toluene (14.2 L) were added and stirred at room temperature for 30 minutes. Next, the solution was cooled to 15° C. and a 50% (w/w) solution of tert-butyldimethylsilyl chloride in toluene (4.372 kg, 14.50 mol) was added in a metered fashion. The reaction was aged for 30 minutes, then sat. aq. NaHCO₃ was added and stirred. The aqueous phase was removed, and the organic residue was washed two times with water. The organic phase was collected, and solvent was removed in vacuo to give 4.330 kg (99% yield) of the title compound as a 50/50 mixture of diastereomers. 1H NMR (600 MHZ, CDCl₃) δ ppm 0.14-0.17 (range, 12H)0.86 (s, 3H)0.89-0.92 (m, 9H)0.96-0.98 (range, 18H)1.02-1.11 (range, 6H) 1.81 (br d, J=6.24 Hz, 6H)1.86-1.95 (m, 2H)2.32-2.40 (m, 2H)3.29 (br s, 1H)3.42 (br s, 1H) 3.74-3.88 (m, 4H)3.93 (br d, J=10.27 Hz, 2H)4.00-4.06 (m, 1H)4.07-4.11 (m, 1H) 4.75-4.97 (m, 5H). GCMS (Cl) m z 301.0 (300.25 calcd. for C₁₇H₃₂O₂Si⁺ [M]⁺).

### Example 4: Preparation of 2-(((tert-butyldimethylsilyl)oxy)methyl)-3,3,4,5-tetramethylhex-5-enal

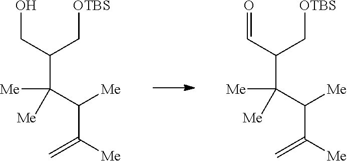 [SMILES: C=C(C)C(C)C(C)(C)C(CO)CO[Si](C)(C)C(C)(C)C ; C=C(C)C(C)C(C)(C)C(C=O)CO[Si](C)(C)C(C)(C)C]

A clean and dry reactor under an air atmosphere was charged with Copper (I) Bromide (353.9 g, 2.46 mol), 2,2′-Bipyridyl (192.1 g, 1.23 mol), 2,2,6,6-Tetramethylpiperidine-1-oxyl free radical (TEMPO) (192.2 g, 1.23 mol), 1-Methylimidazole (101.0 g, 1.23 mol), -(((tert-butyldimethylsilyl)oxy)methyl)-3,3,4,5-tetramethylhex-5-en-1-ol (3.697 kg, 12.30 mol) and Sulfolane (containing 3% water) (11.09 L). The mixture was warmed to 55° C. then sparged with air through a dip tube until reaction deemed complete. Heptane was added and the mixture was washed with 50% (w/w) aq. citric acid, water and 25% (w/w) aq. sodium thiosulfate solution. After removal of the aqueous phase, the organic phase was washed with aq. NaHCO₃, followed by water and sat. aq. sodium chloride. The organic phase was collected, and the solvent removed in vacuo to give 3.329 kg (91% yield) of the title compound. The aldehyde was used in the next step without further purification. GCMS (Cl) m z 299.0 (298.23 calcd. for C₁₇H₃₄O₂Si⁺ [M]⁺).

### Example 5: Preparation of 3,3,4,5-tetramethyl-2-vinylhex-5-en-1-ol

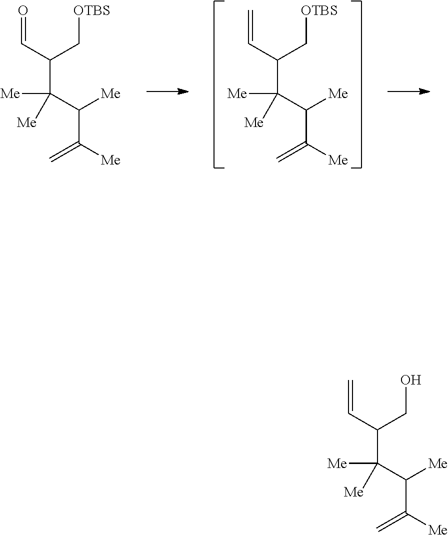

A clean and dry reactor was charged with methyltriphenylphosphonium bromide (4.057 kg, 11.36 mol) then evacuated and backfilled with N₂. THF (6.2 L) was added followed by a 20 wt % solution of potassium tert-butoxide in THF (6.313 kg, 11.25 mol). The mixture was stirred at ambient temperature for 5 hours. The mixture was cooled to −10° C., then 2-(((tert-butyldimethylsilyl)oxy)methyl)-3,3,4,5-tetramethylhex-5-enal (3.082 kg, 10.32 mol) was added. The reaction was stirred at 0° C. until the target conversion was reached. Sat. aq. NaHCO₃ was added followed by the addition of water. Agitation is stopped and the aqueous phase was removed. Heptane, water and methanol were added and agitated. Agitation is stopped and the aqueous phase was removed. The organic phase was subsequently washed twice with a mixture of water and methanol. Next, methanol (12.3 L) and 15% aq. HCl (251 g, 1.03 mol) were added and warmed to 35° C. The reaction was stirred until removal of the TBS ether was deemed complete. The mixture was washed with sat. aq. NaHCO₃, sat. aq. sodium chloride and water. The aqueous phase was removed and the organic phase washed with water. The organic phase was collected, and the solvent removed in vacuo. The crude material was purified by distillation (bp 51° C./0.4 torr) to afford 1.335 kg (71% yield) of the title compound as a mixture of diastereomers. 1H NMR (600 MHz, CDCl₃) δ ppm 0.76-0.84 (m, 9H)0.87 (s, 3H)0.92-0.99 (m, 6H)1.41 (br s, 2H)1.69-1.71 (s, 3H)1.71-1.72 (s, 3H)2.17-2.28 (m, 4H)3.34 (dt, J=15.82, 10.34 Hz, 2H)3.69-3.83 (m, 2H)4.68 (br s, 2H)4.81 (br d, J=13.57 Hz, 2H)5.14 (dd, J=17.06, 1.65 Hz, 2H)5.22-5.29 (m, 2H)5.67-5.75 (m, 2H). GCMS (Cl) m z 182.9 (182.17 calcd. for C₁₂H₂₂O⁺[M]⁺).

### Example 6: Preparation of ((1S,4S)-3,4,5,5-tetramethylcyclopent-2-en-1-yl)methyl isobutyrate (α-necrodyl isobutyrate) from 3,3,4,5-tetramethyl-2-vinylhex-5-en-1-ol

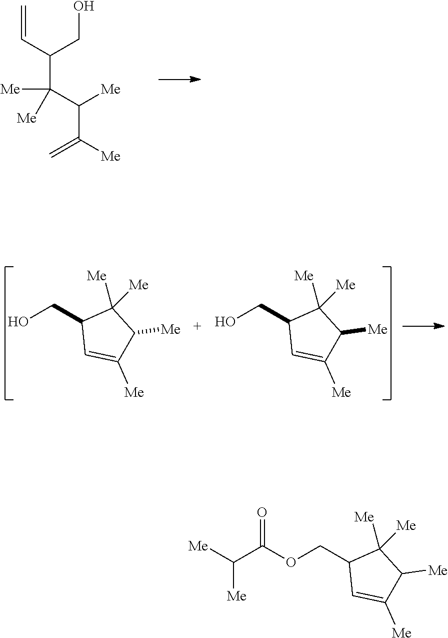

A clean and dry reactor under N₂ atmosphere was charged with 3,3,4,5-tetramethyl-2-vinylhex-5-en-1-ol (1.185 kg, 6.50 mol) and ethyl acetate (3.6 L) then warmed to 60° C. Hoveyda-Grubbs Catalyst (4.07 g, 0.0065 mol) was dissolved in dichloromethane (71 ml) and added to the reactor over 3 hours. The reaction was stirred until deemed complete, then the reaction mixture was cooled to 25° C. and 4-(dimethylamino)pyridine (7.94 g, 0.065 mol) was added followed by isobutyric anhydride (1.182 kg, 7.48 mol). The reaction was stirred until deemed complete. Methanol (234 g, 7.31 mol) was added and stirred for 20 minutes. Next, the reaction was cooled to ambient, and aq. HCl was added. The aqueous phase was removed, then the organic phase was washed with 8% aq. NaHCO₃ two times followed by water. The organic phase was collected, and the solvent removed in vacuo. The crude material was purified by distillation (bp 80° C./2 torr) to afford 1.367 kg (94% yield) of the title compound as a mixture of diastereomers. 1H NMR (600 MHz, CDCl₃) δ ppm 0.79 (s, 3H)0.88-0.93 (m, 6H)0.96-0.99 (m, 6H)1.11 (s, 3H)1.16-1.20 (m, 12H)1.67 (s, 6H)2.11-2.22 (m, 2H)2.48-2.58 (m, 4H)3.96 (ddd, J=15.22, 10.82, 7.15 Hz, 2H)4.04-4.14 (m, 2H)5.15-5.20 (m, 2H). GCMS (Cl) m z 225.0 (224.18 calcd. for C₁₋₄H₂₄O₂⁺ [M]⁺).

### Example 7: Preparation of methyl 3,3,4,5-tetramethylhex-5-enoate

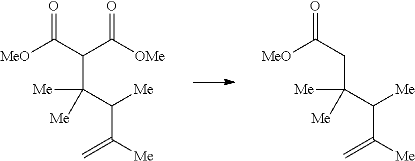 [SMILES: C=C(C)C(C)C(C)(C)C(C(=O)OC)C(=O)OC ; C=C(C)C(C)C(C)(C)CC(=O)OC]

To dimethyl 2-(2,3,4-trimethylpent-4-en-2-yl) malonate (104.00 g, 429.1 mmol) in DMSO (624 mL) was added potassium acetate (210.557 g, 2145.5 mmol) and water (116 mL, 6436.5 mmol). The mixture was stirred at 130° C. until deemed complete. The mixture was cooled to ambient temperature and partitioned between methyl tert-butyl ether and water. The aqueous phase was removed, and the organic phase was washed two times with water. The organic phase was filtered through celite then concentrated in vacuo to give 74.57 g (67% crude yield) of material as an oil. The crude material was purified by distillation (bp 78° C./7 torr) to provide the title compound. GCMS (Cl) m z 184.9 (184.15 calcd. for C₁₁H₂₀O₂⁺ [M]⁺).

### Example 8: Preparation of methyl 2-formyl-3,3,4,5-tetramethylhex-5-enoate

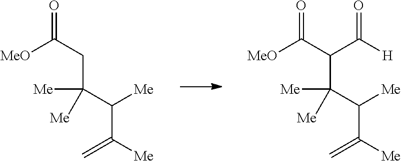

To a solution of diisopropylamine (36.428 g, 360 mmol) in THF (250 mL) at −70° C., n-hexyllithium (2.3 M in hexane, 150 mL, 345 mmol) was added. The reaction was stirred for 20 minutes prior to adding methyl 3,3,4,5-tetramethylhex-5-enoate (51.680 g, 280.4 mmol). The reaction was aged for 90 min. Ethyl formate (41.544 g, 560.8 mmol) was added and stirred until the reaction was deemed complete. A solution of 2N HCl was added and the aqueous phase was removed then the organic material was washed subsequently with sat. aq. NaHCO₃ and water. The organic residue was concentrated to afford 65.54 g (72% yield) of the title compound as an oil. GCMS (Cl) m z 212.9 (212.14 calcd. for C₁₂H₂₀O₃⁺ [M]⁺).

### Example 9: Preparation of methyl 3,3,4,5-tetramethyl-2-vinylhex-5-enoate

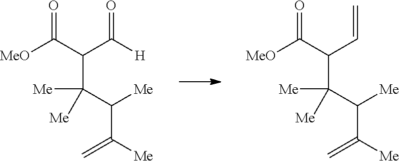

To a solution of methyltriphenylphosphonium bromide (115.028 g, 352 mmol) in THF (300 mL) at 0° C., n-butyllithium (2.5 M in hexanes, 123 mL, 308 mmol) was added. The reaction was aged for 1 hour. The mixture was cooled to −20° C. then methyl 2-formyl-3,3,4,5-tetramethylhex-5-enoate (59.441 g, 280 mmol) was added and stirred while warming to room temperature. When the reaction was deemed complete, 2N HCl was added. The aqueous phase was removed, and the organic phase was subsequently washed with sat. aq. NaHCO₃ and water.

The organic residue was concentrated in vacuo, then heptane was added, and the solution was filtered through silica gel and washed with heptane. The material was concentrated to afford 47.33 g (65% yield) of the title compound as an oil. GCMS (Cl) m z 210.9 (210.16 calcd. for C₁₃H₂₂O₂⁺ [M]⁺).

### Example 10: Preparation of methyl 3,4,5,5-tetramethylcyclopent-2-ene-1-carboxylate

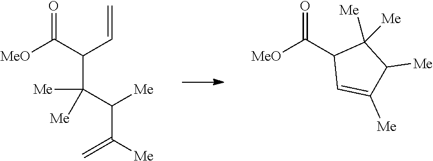

To a solution of methyl 3,3,4,5-tetramethyl-2-vinylhex-5-enoate (238 mg, 1.61 mmol) in toluene (4 mL) was added Hoveyda-Grubbs Catalyst (20.2 mg, 0.032 mmol) and stirred at 30° C. until the reaction was deemed complete. The reaction mixture was filtered through a silica gel plug, eluting with 10% methyl tert-butyl ether in hexanes to afford the title compound as an oil. GCMS (Cl) m z 182.9 (182.13 calcd. for C₁₁H₁₈O₂).

### Example 11: Preparation of 2-(hydroxymethyl)-3,3,4,5-tetramethylhex-5-en-1-yl isobutyrate

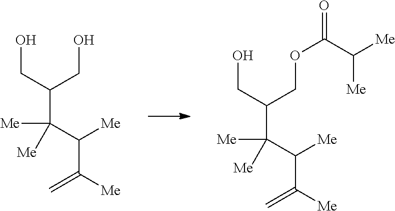 [SMILES: C=C(C)C(C)C(C)(C)C(CO)CO ; C=C(C)C(C)C(C)(C)C(CO)COC(=O)C(C)C]

To a 20 wt % solution of potassium tert-butoxide in THF (621 mL, 1.031 mol), 2-(2,3,4-trimethylpent-4-en-2-yl) propane-1,3-diol (192.14 g, 1.031 mol) was added and stirred at ambient temperature for 30 minutes. A separate clean reactor was charged with isobutyryl chloride (115.38 g, 1.083 mol) and THF (988 mL) then cooled below −20° C. The solution of deprotonated 2-(2,3,4-trimethylpent-4-en-2-yl) propane-1,3-diol was transferred onto to the solution of isobutyryl chloride maintaining the temperature below −20° C. The reaction was stirred while warming to room temperature. Water, 2N HCl and toluene was added. The aqueous phase was removed, and the organic residue was subsequently washed with sat. aq. NaHCO₃ and water. The solvent was removed in vacuo to afford the title compound (262.22 g, 67% yield) as an oil. GCMS (Cl) m z 257.0 (256.20 calcd. for C₁₅H₂₈O₃⁺ [M]⁺).

### Example 12: Preparation of 3,3,4,5-tetramethyl-2-vinylhex-5-en-1-ol

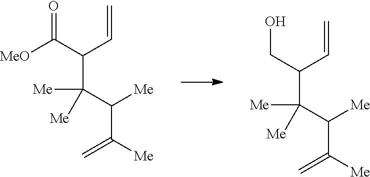

To a solution of methyl 3,3,4,5-tetramethyl-2-vinylhex-5-enoate in toluene at ambient temperature was slowly added a 70% solution of sodium bis(2-methoxyethoxy)aluminum dihydride in toluene (74.7 mL, 289.8 mmol). The reaction was stirred at ambient temperature until deemed complete, then 20% w/w aq. sodium hydroxide was added and stirred until all solids dissolved. The organic phase was washed three times with water. The organic residue was concentrated to afford 32.82 g (74% yield) of the title compound as an oil. GCMS (Cl) m z 182.9 (182.17 calcd. for C₁₂H₂₂O⁺ [M]⁺).

The foregoing description of the specific aspects will so fully reveal the general nature of the technology that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the methods of preparing α-necrodyl isobutyrate are set out in the following numbered embodiments. The first embodiment is denoted E1, the second embodiment is denoted E2 and so forth.

E1. A method of preparing α-necrodyl isobutyrate, the method comprising:
cyclizing a compound of formula (IX):

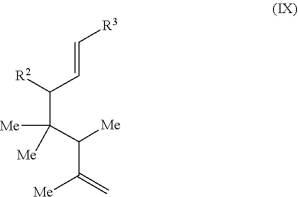

wherein:
R² is

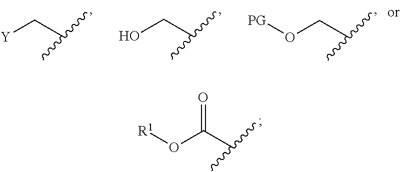

Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl; and

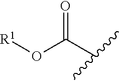

when R² is a reducing agent is added pre-or post-cyclization;
R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents;
R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂;
to produce a mixture of the compounds of formulas (Xa) and (Xb):

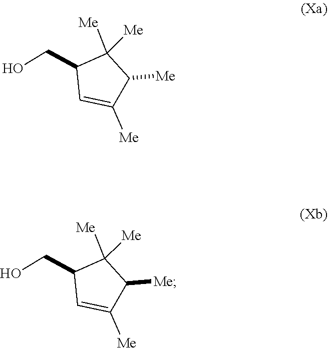 [SMILES: CC1=C[C@@H](CO)C(C)(C)[C@@H]1C ; CC1=C[C@@H](CO)C(C)(C)[C@H]1C]

then
reacting the mixture of compounds of formulas (Xa) and (Xb) with an isobutyryl donor, to produce α-necrodyl isobutyrate; or, alternatively,
cyclizing a compound of formula (IX) wherein R² is

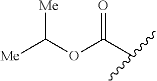 [SMILES: *C(=O)OC(C)C]

to produce
   α-necrodyl isobutyrate.

E2. The method of embodiment 1, wherein cyclizing the compound of formula (IX) comprises reacting the compound of formula (IX) with a transition metal carbene complex catalyst.

E3. The method of embodiment 1 or 2, wherein cyclizing the compound of formula (IX) comprises reacting the compound of formula (IX) with a ruthenium (II) carbene complex catalyst.

E4. The method of embodiment 3, wherein the ruthenium (II) carbene complex catalyst is (1,3-bis(2,4,6-trimethylphenyl)-2-imidazolidinylidene)dichloro(o-isopropoxyphenylmethylene)ruthenium.

E5. The method of any one of embodiments 1-4, wherein R³ is C₁alkyl substituted with 1 substituent selected from the group consisting of

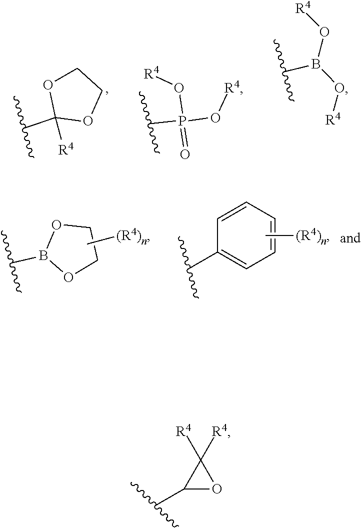

where n is 0, 1, 2, 3, or 4.

E6. The method of any one of embodiments 1-4, wherein R³ is hydrogen.

E7. The method of any one of embodiments 1-6 wherein R² is

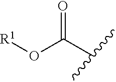

and R¹ is methyl or ethyl.

E8 The method of any one of embodiments 1-6, further comprising olefinating a compound of formula (VII):

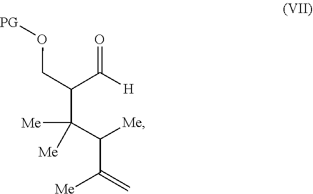

and removing the hydroxyl protecting group (PG), to produce the compound of formula (IX) where R² is

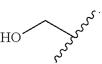 [SMILES: *CO]

E9 The method of any one of embodiments 1-6 or 8, wherein the hydroxyl protecting group is a tert-butyl(dimethyl) silyl (TBS) group.

E10. The method of any one of embodiments 1-6 or 8-9, wherein olefinating the compound of formula (VII) comprises reacting the compound of formula (VII) with a phosphonium ylide of formula (CHR³) PPh₃ or a titanium methylidene of formula (C₅H₅)₂Ti(CHR³).

E11. The method of any one of embodiments 1-6 or 8-10, wherein olefinating the compound of formula (VII) comprises reacting the compound of formula (VII) with a titanium methylidene of formula (C₅H₅)₂Ti(CH₂).

E12. The method of any one of embodiments 1-6 or 8-10, wherein olefinating the compound of formula (VIII) comprises reacting the compound of formula (VII) with a phosphonium ylide of formula (CH₂) PPh₃.

E13. The method of any one of embodiments 1-6 or 8-12, further comprising oxidizing a compound of formula (VI):

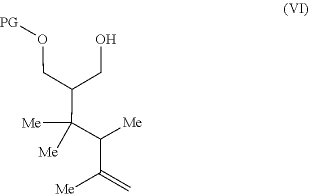

to produce the compound of formula (VII).

E14. The method of any one of embodiments 1-6 or 8-13, wherein oxidizing the compound of formula (IV) comprises reacting the compound of formula (IV) with a nitroxyl radical, a hypervalent iodine compound or activated DMSO.

E15. The method of any one of embodiments 1-6 or 8-14, wherein oxidizing the compound of formula (IV) comprises reacting the compound of formula (IV) with a nitroxyl radical.

E16. The method of embodiment 15, wherein the nitroxyl radical is formed by reacting a nitroxyl radical precursor with a stoichiometric oxidant, where the nitroxyl radical precursor is:
(2,2,6,6-tetramethylpiperidin-1-yl)oxyl (TEMPO),
4-hydroxy-(2,2,6,6-tetramethylpiperidin-1-yl)oxyl4-acetamido (4-OH-TEMPO),
4-acetamido-(2,2,6,6-tetramethylpiperidin-1-yl)oxyl (4-acetamido-TEMPO),
2-azaadamantane N-oxyl (AZADO), or 9-azabicyclo[3.3.1]nonane N-oxyl (ABNO).

E17. The method of embodiment 16, wherein the nitroxyl radical precursor is TEMPO.

E18. The method of embodiment 16 or 17, wherein the stoichiometric oxidant is sodium hypochlorite, oxygen, or (diacetoxyiodo)benzene.

E19. The method of any one of embodiments 16-18, wherein the stoichiometric oxidant is sodium hypochlorite.

E20. The method of any one of embodiments 1-6 or 8-19, further comprising adding a hydroxyl protecting group to the compound of formula (V):

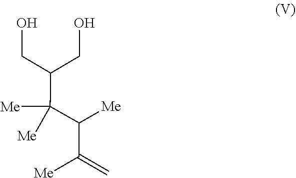

to produce the compound of formula (VI).

E21. The method of any one of embodiments 1-6 or 8-20, further comprising reducing the compound of formula (III):

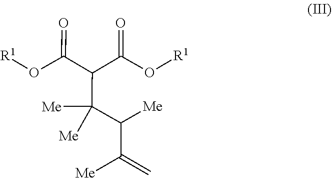

with a reducing agent to produce the compound of formula (V).

E22. The method of any one of embodiments 1-21, wherein the reducing agent is a metal hydride.

E23. The method of any one of embodiments 1-22, wherein the reducing agent is lithium aluminum hydride (LiAlH₄), sodium borohydride (NaBH₄), sodium bis(2-methoxyethoxy)aluminum hydride, or diisobutylaluminum hydride (DIBAL-H).

E24. The method of any one of embodiments 1-23, wherein the reducing agent is sodium bis(2-methoxyethoxy)aluminum hydride.

E25. The method of any one of embodiments 1-7, further comprising olefinating a compound of formula (VIII):

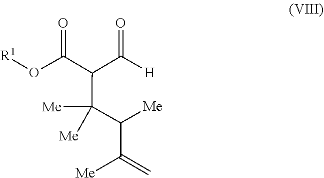

to produce the compound of formula (IX) where R² is

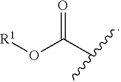

E26. The method of any one of embodiments 1-7 or 25, wherein olefinating the compound of formula (VIII) comprises reacting the compound of formula (VIII) with a phosphonium ylide of formula (CHR³) PPh₃ or a titanium methylidene of formula (C₅H₅)₂Ti(CHR³).

E27. The method of any one of embodiments 1-7 or 25-26, wherein olefinating the compound of formula (VIII) comprises reacting the compound of formula (VIII) with a titanium methylidene of formula (C₅H₅)₂Ti(CH₂).

E28. The method of any one of embodiments 1-7 or 25-26, wherein olefinating the compound of formula (VIII) comprises reacting the compound of formula (VIII) with a phosphonium ylide of formula (CH₂) PPh₃.

E29. The method of any one of embodiments 1-7 or 25-28, further comprising formylating the compound of formula (IV):

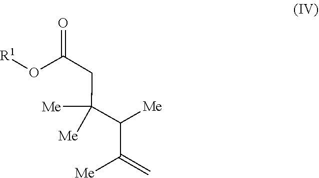

to produce the compound of formula (VIII).

E30. The method of any one of embodiments 1-7 or 23-29, further comprising selectively decarboxylating one

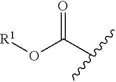

moiety in the compound of formula (III):

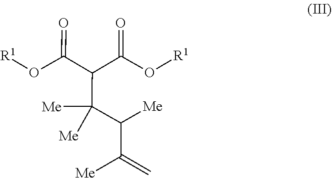

to produce the compound of formula (IV).

E31. The method of any one of embodiments 1-7 or 23-29, wherein decarboxylating one

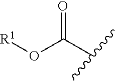

moiety in the compound of formula (III) comprises reacting the compound of formula (III) with LiCl.

E32. The method of any one of embodiments 1-31, further comprising reacting a compound of formula (I) wherein X is chlorine, bromine or iodine, with a compound of formula (II):

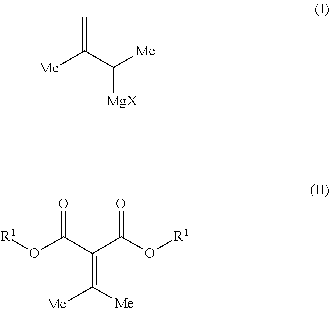

to produce the compound of formula (III).

E33. A compound of formula (IX):

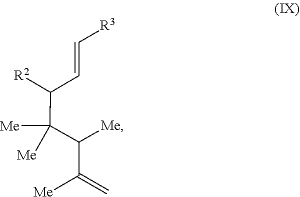

wherein:
R² is

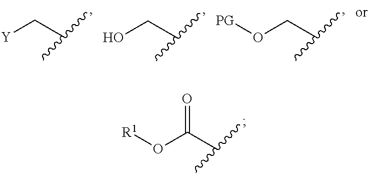

Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.
R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, —C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents; and
R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂.

E34. The compound of embodiment 33, wherein R³ is C₁alkyl substituted with 1 substituent selected from the group consisting of

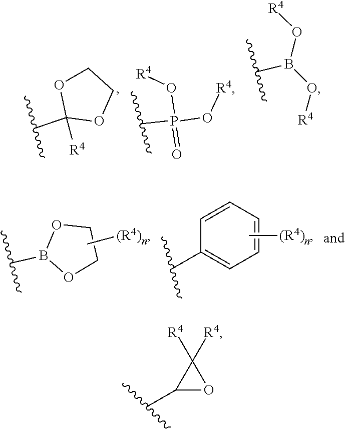

where n is 0, 1, 2, 3, or 4.

E35. The compound of embodiment 33, wherein R³ is hydrogen.

E36. A compound of formula (XI):

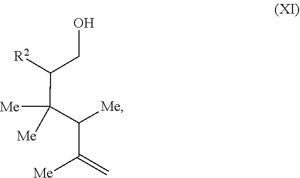

wherein:
R² is

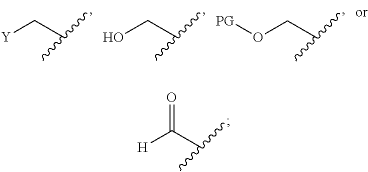

and
Y is a halogen;
PG is a hydroxyl protecting group.

E37. The compound of embodiment 36, wherein the hydroxyl protecting group is a tert-butyl(dimethyl) silyl (TBS) group.

E38. A compound of formula (VIII):

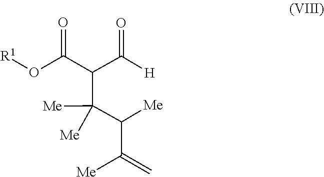

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E39. A compound of formula (IV):

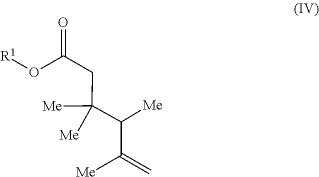

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E40. A compound of formula (III):

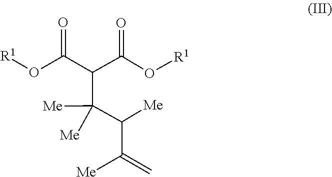

wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

E41. The compound of any one of embodiments 33-34 or 38-40 wherein R¹ is methyl or ethyl.

1 IX. A method of preparing α-necrodyl isobutyrate, the method comprising:
cyclizing a compound of formula (IX): 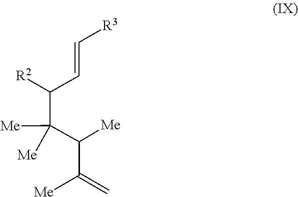
wherein: 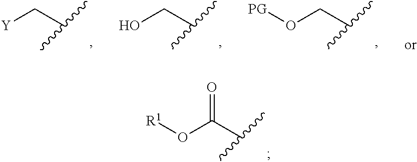
R² is
Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl; and
when R² is 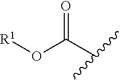
a reducing agent is added pre-or post-cyclization;
R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, —C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents;
R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂;
to produce a mixture of the compounds of formulas (Xa) and (Xb): 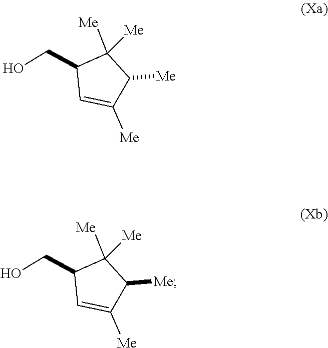 [SMILES: CC1=C[C@@H](CO)C(C)(C)[C@@H]1C ; CC1=C[C@@H](CO)C(C)(C)[C@H]1C]
then
reacting the mixture of compounds of formulas (Xa) and (Xb) with an isobutyryl donor, to produce α-necrodyl isobutyrate; or, alternatively,
cyclizing a compound of formula (IX) wherein R² is 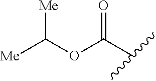 [SMILES: *C(=O)OC(C)C]
to produce α-necrodyl isobutyrate.

2. The method of claim 1, wherein cyclizing the compound of formula (IX) comprises reacting the compound of formula (IX) with a transition metal carbene complex catalyst.

3. The method of claim 1 or 2, wherein cyclizing the compound of formula (IX) comprises reacting the compound of formula (IX) with a ruthenium (II) carbene complex catalyst.

4. The method of claim 3, wherein the ruthenium (II) carbene complex catalyst is (1,3-bis(2,4,6-trimethylphenyl)-2-imidazolidinylidene)dichloro(o-isopropoxyphenylmethylene)ruthenium.

5. The method of any one of claims 1-4, wherein R³ is C₁alkyl substituted with 1 substituent selected from the group consisting of 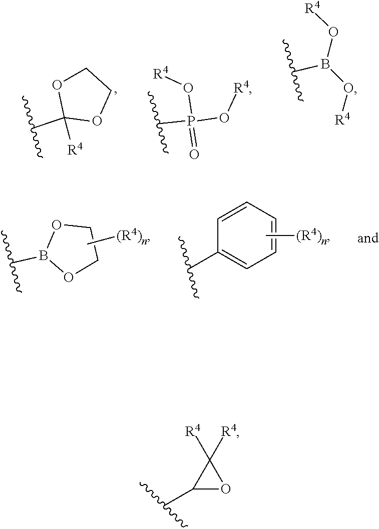
where n is 0, 1, 2, 3, or 4.

6. The method of any one of claims 1-4, wherein R³ is hydrogen.

7. The method of any one of claims 1-6 wherein R² is 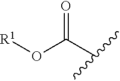
and R¹ is methyl or ethyl.

8. The method of any one of claims 1-6, further comprising olefinating a compound of formula (VII): 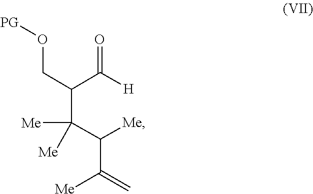
and removing the hydroxyl protecting group (PG), to produce the compound of formula (IX) where R² is

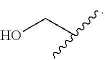 [SMILES: *CO]

9. The method of any one of claim 1-6 or 8, wherein the hydroxyl protecting group is a tert-butyl(dimethyl) silyl (TBS) group.

10. The method of any one of claim 1-6 or 8-9, wherein olefinating the compound of formula (VII) comprises reacting the compound of formula (VII) with a phosphonium ylide of formula (CHR³)PPh₃ or a titanium methylidene of formula (C₅H₅)₂Ti(CHR³).

11. The method of any one of claim 1-6 or 8-10, wherein olefinating the compound of formula (VII) comprises reacting the compound of formula (VII) with a titanium methylidene of formula (C₅H₅)₂Ti(CH₂).

12. The method of any one of claim 1-6 or 8-10, wherein olefinating the compound of formula (VIII) comprises reacting the compound of formula (VII) with a phosphonium ylide of formula (CH₂) PPh₃.

13. The method of any one of claim 1-6 or 8-12, further comprising oxidizing a compound of formula (VI): 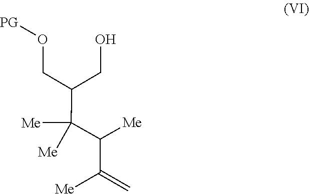
to produce the compound of formula (VII).

14. The method of any one of claim 1-6 or 8-13, wherein oxidizing the compound of formula (IV) comprises reacting the compound of formula (IV) with a nitroxyl radical, a hypervalent iodine compound or activated DMSO.

15. The method of any one of claim 1-6 or 8-14, wherein oxidizing the compound of formula (IV) comprises reacting the compound of formula (IV) with a nitroxyl radical.

16. The method of claim 15, wherein the nitroxyl radical is formed by reacting a nitroxyl radical precursor with a stoichiometric oxidant, where the nitroxyl radical precursor is (2,2,6,6-tetramethylpiperidin-1-yl)oxyl (TEMPO),
4-hydroxy-(2,2,6,6-tetramethylpiperidin-1-yl)oxyl4-acetamido (4-OH-TEMPO),
4-acetamido-(2,2,6,6-tetramethylpiperidin-1-yl)oxyl (4-acetamido-TEMPO),
2-azaadamantane N-oxyl (AZADO), or 9-azabicyclo[3.3.1]nonane N-oxyl (ABNO).

17. The method of claim 16, wherein the nitroxyl radical precursor is TEMPO.

18. The method of claim 16 or 17, wherein the stoichiometric oxidant is sodium hypochlorite, oxygen, or (diacetoxyiodo)benzene.

19. The method of any one of claims 16-18, wherein the stoichiometric oxidant is sodium hypochlorite.

20. The method of any one of claim 1-6 or 8-19, further comprising adding a hydroxyl protecting group to the compound of formula (V): 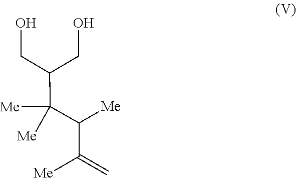
to produce the compound of formula (VI).

21. The method of any one of claim 1-6 or 8-20, further comprising reducing the compound of formula (III): 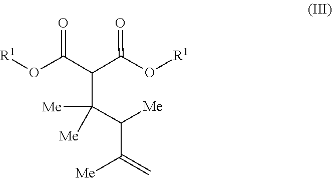
with a reducing agent to produce the compound of formula (V).

22. The method of any one of claims 1-21, wherein the reducing agent is a metal hydride.

23. The method of any one of claims 1-22, wherein the reducing agent is lithium aluminum hydride (LiAlH₄), sodium borohydride (NaBH₄), sodium bis(2-methoxyethoxy)aluminum hydride, or diisobutylaluminum hydride (DIBAL-H).

24. The method of any one of claims 1-23, wherein the reducing agent is sodium bis(2-methoxyethoxy)aluminum hydride.

25. The method of any one of claims 1-7, further comprising olefinating a compound of formula (VIII): 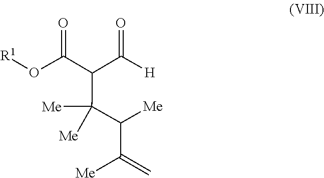
to produce the compound of formula (IX) where R² is

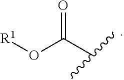

26. The method of any one of claim 1-7 or 25, wherein olefinating the compound of formula (VIII) comprises reacting the compound of formula (VIII) with a phosphonium ylide of formula (CHR³) PPh₃ or a titanium methylidene of formula (C₅H₅)₂Ti(CHR³).

27. The method of any one of claim 1-7 or 25-26, wherein olefinating the compound of formula (VIII) comprises reacting the compound of formula (VIII) with a titanium methylidene of formula (C₅H₅)₂Ti(CH₂).

28. The method of any one of claim 1-7 or 25-26, wherein olefinating the compound of formula (VIII) comprises reacting the compound of formula (VIII) with a phosphonium ylide of formula (CH₂)PPh₃.

29. The method of any one of claim 1-7 or 25-28, further comprising formylating the compound of formula (IV): 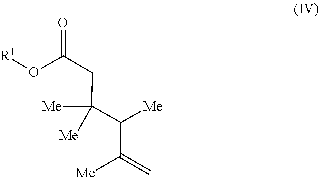
to produce the compound of formula (VIII).

30. The method of any one of claim 1-7 or 23-29, further comprising selectively decarboxylating one 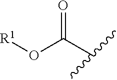
moiety in the compound of formula (III): 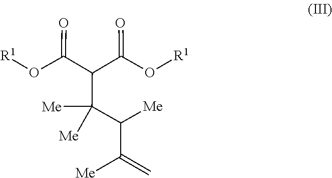
to produce the compound of formula (IV).

31. The method of any one of claim 1-7 or 23-29, wherein decarboxylating one 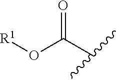
moiety in the compound of formula (III) comprises reacting the compound of formula (III) with LiCl.

32. The method of any one of claims 1-31, further comprising reacting a compound of formula (I), wherein X is chlorine, bromine, or iodine, with a compound of formula (II): 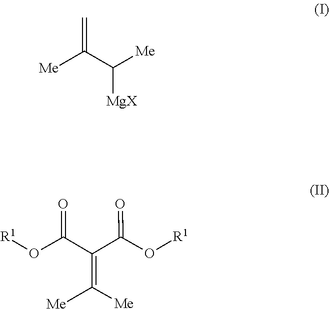
to produce the compound of formula (III).

33. A compound of formula (IX): 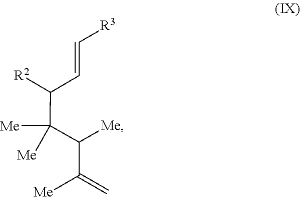
wherein:
R² is 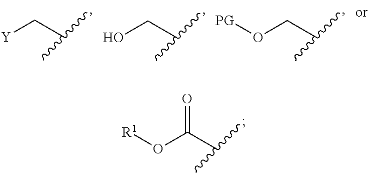
Y is a halogen;
PG is a hydroxyl protecting group;
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl;
R³ is hydrogen, C₁₋₁₀alkyl, or C₁₋₁₀alkyl substituted with 1-4 substituents independently selected from the group consisting of halogen, —OR⁴, —N(R⁴)₂, —CO₂R⁴, —C(O)R⁴, —Si(R⁴)₃, —B(OR⁴)₂, a 3-to 12-membered heterocycle optionally substituted with 1-4 R⁴ substituents, and a 3-to 12-membered carbocycle optionally substituted with 1-4 R⁴ substituents; and
R⁴ at each occurrence, is independently hydrogen, halogen, C₁₋₄alkyl, —OH, —OC₁₋₄alkyl, —NH₂, —NHC₁₋₄alkyl, —N(C₁₋₄alkyl)₂, —C(O)C₁₋₄alkyl, —CO₂C₁₋₄alkyl, —C(O) H, —CO₂H, —C(O)NC₁₋₄alkyl, —CF₃, —CHF₂, or —NO₂.

34. The compound of claim 33, wherein R³ is C₁alkyl substituted with 1 substituent selected from the group consisting of 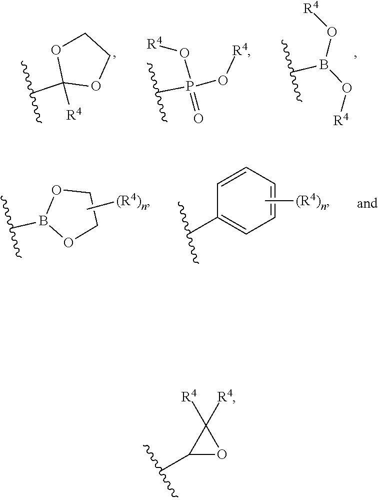
where n is 0, 1, 2, 3, or 4.

35. The compound of claim 33, wherein R³ is hydrogen.

36. A compound of formula (XI): 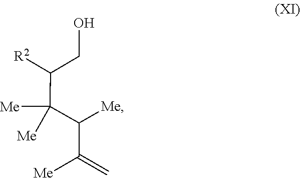
wherein:
R² is 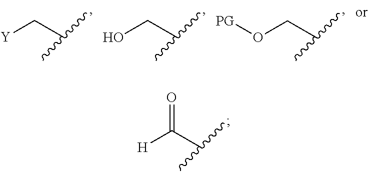
and
Y is a halogen;
PG is a hydroxyl protecting group.

37. The compound of claim 36, wherein the hydroxyl protecting group is a tert-butyl(dimethyl) silyl (TBS) group.

38. A compound of formula (VIII): 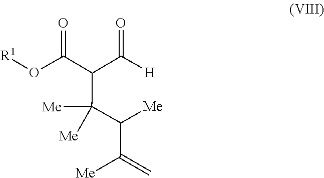
wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

39. A compound of formula (IV): 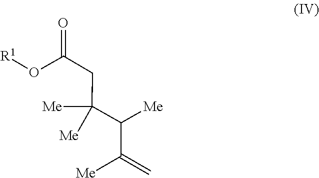
wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

40. A compound of formula (III): 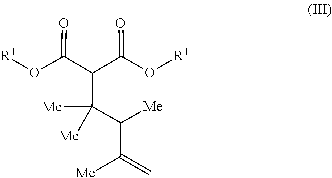
wherein:
R¹ is C₁₋₁₀alkyl, C₃₋₇cycloalkyl, a 6-to 12-membered aryl, or hydrogen, wherein the C₁₋₁₀alkyl, the C₃₋₇cycloalkyl, and the 6-to 12-membered aryl are each optionally substituted with 1-5 substituents independently selected from the group consisting of C₁₋₄alkyl, C₁₋₂haloalkyl, halogen, cyano, —OC₁₋₄alkyl, and —OC₁₋₂haloalkyl.

41. The compound of any one of claim 33-34 or 38-40 wherein R¹ is methyl or ethyl.
